SEQUENCE LISTING (9 sequences)

SEQ ID NO: 1 - protein, 28 aa, Artificial Sequence
  Peptide encoded by M13 clone 1
PGGFRGEFMLGKPDPKPEGKGLGSPYIE

SEQ ID NO: 2 - protein, 121 aa, Artificial Sequence
  Peptide encoded by M13 clone 2
ADDNFSIPEGEEDLAKAIQMAQEQATDTEILERKTVLPSKHAVPEVIEDFLCNFLIKMGMTRTLDCFQSEWYELIQKGVTELRTVGNVPDVYTQIMLLENENKNLKKDLKHYKQAAEYVIF

SEQ ID NO: 3 - protein, 112 aa, Artificial Sequence
  GeneVI Figure2
MPVLLGIPLLLRFLGFLLVTLFGYLLTFLKKGFGKIAIAISLFLALIIGLNSILVGYLSDISAQLPSDFVQGVQLILPSNALPCFYVILSVKAAIFIFDVKQKIVSYLDWDK

SEQ ID NO: 4 - protein, 17 aa, Artificial Sequence
  GS linker Figure2
GSGGGSGGGPSRPDLLE

SEQ ID NO: 5 - protein, 12 aa, Artificial Sequence
  WT M13 Figure2
NSSSRVPRPLIN

SEQ ID NO: 6 - protein, 41 aa, Artificial Sequence
  M13 clone 1 figure 2
LVDPPGCRNSARGPGGFRGEFMLGKPDPKPEGKGLGSPYIE

SEQ ID NO: 7 - protein, 126 aa, Artificial Sequence
  M13 clone 2 figure 2
NSARGADDNFSIPEGEEDLAKAIQMAQEQATDTEILERKTVLPSKHAVPEVIEDFLCNFLIKMGMTRTLDCFQSEWYELIQKGVTELRTVGNVPDVYTQIMLLENENKNLKKDLKHYKQAAEYVIF

SEQ ID NO: 8 - protein, 18 aa, Artificial Sequence
  WT T7 figure 2
MLGDPNSSSVDKLAAALE

SEQ ID NO: 9 - protein, 26 aa, Artificial Sequence
  T7 S-Tag figure 2
MLGDPNKETAAAKFERQHMDSLAALE

(19) United States
(12) Patent Application Publication
(10) Pub. No.: US 2012/0322981 A1
(43) Pub. Date: Dec. 20, 2012

(54) CITRULLINATION-SPECIFIC PHAGE DISPLAY

(76) Inventors: Veerle Somers, Sint-Truiden (BE); Klaartje Somers, Vliermaalroot (BE); Piet Stinissen, Diepenbeek (BE)

(21) Appl. No.: 13/514,953

(22) PCT Filed: Dec. 7, 2010
(86) PCT No.: PCT/EP10/69034

§ 371 Date: Aug. 22, 2012

(30) Foreign Application Priority Data
Dec. 8, 2009 (EP) 09178406.6

(51) Int. Cl. C12N 7/00; C07K 1/14; C07K 16/00

(52) U.S. Cl. 530/387.1; 435/235.1

(57) ABSTRACT

The invention relates to a modified phage display that allows the specific detection of citrullinated proteins. More specifically, the invention relates to a method for citrullinating proteins displayed by phage, without losing phage infectivity, and the detection of those proteins by biopanning. In a preferred embodiment, the phage is a T7 phage.

### CROSS-REFERENCE TO RELATED APPLICATIONS

This is a national phase entry under 35 U.S.C. §371 of International Patent Application PCT/EP2010/069034, filed Dec. 7, 2010, published in English as International Patent Publication WO 2011/069993 A1 on Jun. 16, 2011, which claims the benefit under Article 8 of the Patent Cooperation Treaty to European Patent Application Serial No. 09178406.6, filed Dec. 8, 2009.

### TECHNICAL FIELD

The invention relates to a modified phage display that allows the specific detection of citrullinated proteins. More specifically, the invention relates to a method for citrullinating proteins displayed by phage, without losing phage infectivity, and the detection and selection of those proteins by biopanning. In a preferred embodiment, the phage is a T7 phage.

### BACKGROUND

More than 20 years ago, phage display technology was developed by Smith (1985). The technique is based on the ability of phage virions, virus particles that infect and amplify in bacteria, to incorporate foreign DNA into their genome, coupled to a gene encoding a phage coat protein (Smith, 1985; Webster, 1996). After infection, phage protein components are produced by the protein translation machinery of the infected bacterial host cell and the incorporated DNA is translated into the corresponding DNA product, covalently coupled to the phage coat protein. Upon phage virion assembly, the recombinant coat protein will be incorporated into the virion protein coat (Webster, 1996). The peptide/protein product, encoded by the DNA insert, is displayed at the surface of the phage particle and is thus available for experimental strategies. The strength of the phage display technology lies within the physical link between DNA and DNA product (through the protein coat of the virus), which allows for the succession of affinity selection and amplification of selected phage particles resulting in powerful enrichment of selected phage and an increase in assay sensitivity.

Different phage display systems have been developed throughout the years, making use of different phage vectors (M13 filamentous phage, lambda, T4 and T7 phage) and various phage coat proteins for covalent fusion. The M13 filamentous phage is employed most commonly. The strength of the filamentous phage display system lies within the lysogenic life cycle of this phage and the availability of M13 phagemid vectors (Webster, 1006; Hufton et al., 1999). Lysogenic phage integrate their DNA into the host cell genome, are replicated along with the bacterial cell and do not require the lysis of the bacterial cell for phage particle formation. Instead, phage particles are shed from the bacterial surface without inducing cell death (Webster, 1996). Moreover, the development of M13 phagemid vectors has allowed for excellent workability. Phagemids are plasmids containing the replication origin and packaging signal of the filamentous phage, together with the plasmid origin of replication and the gene encoding the phage coat protein coupled with the DNA insert (Webster, 1996; Armstrong et al., 1996). For phage propagation, bacterial cells infected with phagemid need to be “superinfected” with a so-called helper phage that provides all the other essential phage components for the formation of viable phage virions. Besides excellent workability, the use of a phagemid vector system allows for monovalent display of the recombinant protein (maximally one recombinant protein per phage virion) as the helper phage contributes non-recombinant phage coat proteins (Armstrong et al., 1996). Different M13 vector systems for phage display through various coat proteins are available (Smith and Petrenko, 1997; Barbas, 1993). Major coat protein pVIII and minor coat protein pIII, are used most frequently for display purposes (Armstrong et al., 1996; Rodi and Makowski, 1999). As the N-terminal end of both proteins is exposed at the phage surface, foreign DNA sequences are inserted upstream of the genes encoding the coat proteins. The development of phage vectors for C-terminal fusion to M13 minor coat protein pVI, of which the C-terminal end is exposed at the surface of the phage, has been an important step towards the development of cDNA phage display libraries (Hufton et al., 1999; Jespers et al., 1995).

More recently, display methods were also developed in the lytic phage systems, namely for lambda phage, T4 and T7 phage. For the formation and shedding of recombinant lytic phage virions containing recombinant coat proteins, bacterial cells need to be lysed on phage propagation (Russel, 1991). Moreover, as there are no plasmid vectors available for the lytic phage, DNA isolation and experimental approaches are more labor-intensive in comparison to working with plasmids (Sambrook et al., 1989). As both lytic and lysogenic phage life cycles employ different phage assembly strategies, both approaches allow the display of different proteins (Hufton et al., 1999). As the virion proteins of M13 filamentous phage (and thus, also the recombinant phage coat protein) are embedded into the bacterial cell membrane prior to phage virion assembly, this process puts constraints on the proteins that can be displayed at the surface of the phage; for efficient display, the cDNA products must be able to traverse the bacterial cell membrane and need to allow for the formation of a viable and infectious virion (Webster, 1996; Russel, 1991; Rodi et al., 2002). For lytic phage virion production on the other hand, the recombinant proteins are formed and retained within the cytosol of bacterial cells prior and during virion assembly so that the spectrum of recombinant proteins that can be displayed by lytic phage is less constrained (Hufton et al., 1999; Russel, 1991; Krumpe et al., 2006).

Phage display is a powerful technology used for identifying interacting molecules and ligands for a given target. The technique has a broad range of applications, such as drug and target discovery, protein evolution and rational drug design. Phage particles are amenable to the display of entire peptide libraries, both constrained (cyclic) or unconstrained, antibody fragment libraries (Marks et al., 1991; McCafferty et al., 1990), enzymes (Soumillion et al., 1994), genomes (Jacobsson et al., 2003) and entire, fractionated or full-length, cDNA libraries (Crameri et al., 1994). In this way, the technique has proven to be useful in different domains, such as in the identification of peptide ligands for various targets (as a mimic for peptides/proteins or even carbohydrates and lipids, called peptidomimetics), in epitope mapping, in the development of antibody specificities with increased affinity for a particular ligand and in the elucidation of the substrates targeted by enzymes (Smith and Petrenko, 1997).

Despite the obtained successes of phage display technology in biochemistry, cancer and immunology research, the main drawback of the technique is the use of the bacterial protein translational machinery for the production of phage virion proteins including the recombinant coat protein. A major difference between prokaryotic and eukaryotic protein translation systems is the potential introduction of post-translational modifications (PTMs) in proteins of eukaryotic species. PTMs of proteins such as glycosylation and phosphorylation, play a role in protein functioning and are essential in normal physiological conditions (Alberts et al., 2008). It is, thus, not surprising that aberrant PTMs have been associated with different diseases such as cancer and autoimmunity (Krueger and Srivastava, 2006; Anderton, 2004). To this end, PTMs can be important in the identification of ligands for specific targets.

Due to the importance of the PTMs, several phage display systems have been developed to detect modified proteins. Panning with in vitro phosphorylated phage has been described for M13/pVIII (Schmitz at al., JMB 260:664-677, 1996; Dente et al, 269:694-703, 1997). Stolz et al. (FEBS Lett. 440:213-217, 1998) describe the (in vivo) biotinylation of proteins displayed on bacteriophage lambda. U.S. Pat. No. 7,141,366 (New England Biolabs) describes a surface display system where selenocysteine is incorporated in the sequence, whereby this amino acid further can be modified.

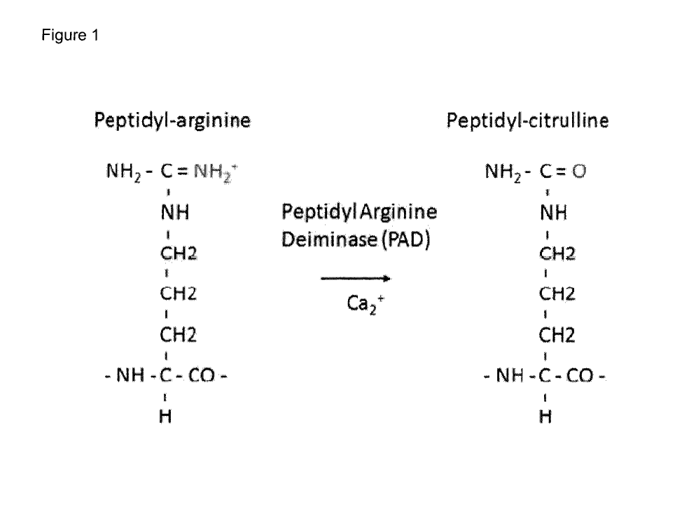

Citrullination, which is the post-translational modification of an arginine amino acid into a citrulline amino acid by peptidyl arginine deiminase (PAD) enzymes (FIG. 1), is one of the PTMs currently focused on in different research domains. During recent years, this PTM has become of increasing interest and is shown to be involved in several physiological processes including terminal differentiation of the epidermis (Mechin et al., 2005; Nachat et al., 2005), apoptosis and gene regulation (Asaga et al., 1998; Li et al., 2008; Yao et al., 2008). Furthermore, citrullination has now also moved into the focus of research on several diseases such as multiple sclerosis (Mastronardi et al., 2006; Musse et al., 2006; Deraos et al., 2008; Nicholas et al., 2004; Raijmakers et al., 2005), Alzheimer's disease (Ishigami et al., 2005), psoriasis (Ishida-Yamamoto et al., 2000) and especially, rheumatoid arthritis (RA) (Schellekens et al., 1998; van Boekel and van Venrooij, 2003). These findings indicate the need for a highly sensitive, high-throughput approach for the identification of citrullinated proteins, allowing, as a non-limiting example, the elucidation of the complexity of the RA synovial citrullinome so that more can be learned about its involvement in the pathology and etiology of the disease. Despite its importance, no phage display system for citrullinated proteins has been described. The effecting of citrullination is expected to have a stronger affect on structure and biological activity of the protein that is displayed than phosphorylation, and the techniques applied for other PTMs cannot be applied to citrullination without undue experimentation. Indeed, introduction of citrulline dramatically changes the structure and function of proteins (György et al., 2006) by inducing protein unfolding (Tarsca et al., 1996).

### DISCLOSURE

Surprisingly, and contrary to what would be expected by the person skilled in the art, knowing the protein denaturing effect of a peptidyl arginine deiminase treatment (Tarsca et al., 1996), we found that it is possible to citrullinate a protein, presented by a phage, without losing the infectivity of the phage.

Described is an infective phage displaying a peptide whereby at least one arginine of the displayed peptide is citrullinated. “Infective,” as used herein, means that the phage is still able to adhere to the host cell, to transfer its genetic material to the host cell and to replicate in the host. A citrullinated phage is considered as infective if, after citrullination, it keeps 20%, preferably 30%, more preferably 40%, more preferably 50%, more preferably 60%, more preferably 70%, even more preferably 80%, most preferably 90% of the infective capacity of the wild-type, as expressed in plaque- or colony-forming units per ml. “Peptide,” as used herein, is referring to a polymer of amino acids and does not refer to a specific length of the molecule. Phages used for phage display are known to the person skilled in the art and include, but are not limited to T4, T7, Lambda and M13. Preferably, the phage is T7. Preferably, the citrullination is carried out in vitro, on one or more peptides displaying phage. To improve the infectivity of the phage after citrullination, arginine residues in phage proteins, which are important for the phage-host interaction, may be replaced by other amino acids, preferably by other polar amino acids, even more preferably by other positively charged amino acids.

Also described is the use of a phage displaying a citrullinated peptide, according to the invention, to isolate polypeptides binding citrullinated proteins. “Binding” means any interaction, be it direct or indirect. A direct interaction implies a contact between the binding partners. An indirect interaction means any interaction whereby the interaction partners interact in a complex of more than two compounds. The interaction can be completely indirect, with the help of one or more bridging molecules, or partly indirect, where there is still a direct contact between the partners, which is stabilized by the additional interaction of one or more compounds. The terms “protein” and “polypeptide” as used in this application are interchangeable. “Polypeptide” refers to a polymer of amino acids and does not refer to a specific length of the molecule. This term also includes post-translational modifications of the polypeptide, such as glycosylation, phosphorylation and acetylation. Preferably, the polypeptide is an antibody directed against citrullinated peptides and proteins (APCAs). It is clear for the person skilled in the art that the phage according to the invention can also be used to map the epitopes of the APCAs. Preferably, the APCAs are RA autoantibodies. Indeed, studies in rheumatoid arthritis (RA) have shown that citrullination as PTM plays a role in the escape of self-tolerance and, thus, potentially lies at the basis of the RA pathogenesis, at least in a subgroup of RA patients (van Venrooij et al., 2008; Hill et al., 2003). Citrullination has been shown to occur in inflammatory conditions and citrullinated proteins have been detected in synovial joints of patients with various inflammatory diseases (Vossenaer et al., 2004; Lundberg et al., 2005; Chapuy-Regaud et al., 2005; Cantaert et al., 2006). However, the development of antibodies directed against these citrullinated proteins (ACPA), is specifically associated with RA. A citrullinated peptide library or citrullinated RA synovium cDNA expression library displayed at the surface of phage particles, preferably T7 phage particles, can be used for high-throughput and highly sensitive epitope mapping of the ACPA antibodies: affinity selection of a citrullinated phage display library with pooled purified ACPA (isolated from RA patients), pooled anti-CCP antibody-positive RA serum or monoclonal antibodies mimicking particular ACPA antibody specificities is useful for the identification of high-affinity ACPA ligands, which can be applied in novel serological ACPA tests. Moreover, the citrullination of an entire RA synovium expression library displayed on phage, preferably T7 phage, will allow for the highly sensitive identification of all possible in vivo citrullinated targets and will provide important clues as to which synovial citrullinated proteins are essential to the induction and perpetuation of the ACPA response.

As recent reports propose a possible role of citrullination in multiple sclerosis, psoriasis and Alzheimer's disease, as mentioned above, the potential use of the phage displaying a citrullinated protein extends to these research domains as well.

Still also described is a method to citrullinate a peptide-displaying phage, without affecting the infective capacity of the phage, resulting in an infective phage, displaying a citrullinated peptide, according to the invention. “Without affecting the infective capacity,” as used herein, means that the citrullinated phage keeps 20%, preferably 30%, more preferably 40%, more preferably 50%, more preferably 60%, more preferably 70%, even more preferably 80%, most preferably 90% of the infective capacity of the wild-type, as expressed in plaque- or colony-forming units per ml. Preferably, the phage is a T7 phage. Preferably, the citrullination is carried out in vitro. Even more preferably, the citrullination is carried out by treatment of the peptide-displaying phage with a Ca²⁺-dependent peptidyl arginine deaminase.

### BRIEF DESCRIPTION OF THE FIGS.

FIG. 1: Enzymatic conversion reaction of an arginine amino acid into a citrulline amino acid. Ca2+-dependent peptidyl arginine deiminase (PAD) enzymes convert positively charged arginine into a neutral citrulline by a deimination reaction. We tested whether citrullination as a PTM could be implemented in phage display by performing in vitro citrullination and infectivity experiments with two different phage display systems, namely, the M13 filamentous and T7 lytic phage display systems. We show for the first time that citrullination can efficiently be achieved in vitro in T7 phage particles and their displayed peptides/proteins without loss of viability and infectivity. The possibility to achieve in vitro citrullination in T7 phage particles allows for the implementation of T7 phage display systems in approaches aimed at the identification of citrulline-containing ligands.

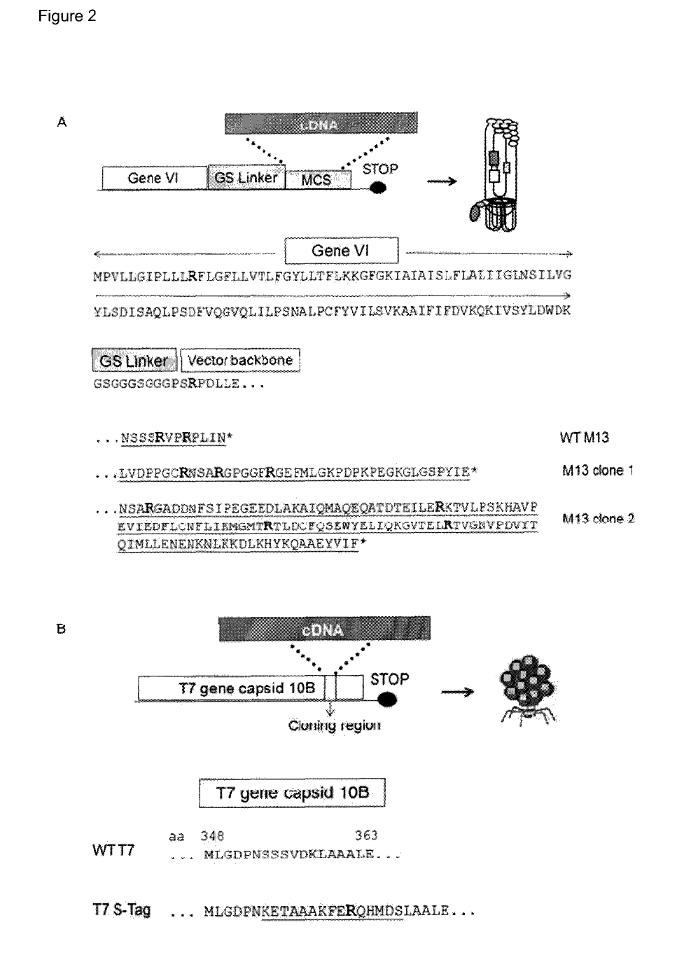

FIG. 2: M13 and T7 phage display vectors used for citrullination experiments. (A) M13 pVI-display phagemid vector containing a multiple cloning site (MCS) at the 3′ end of the gene encoding minor phage coat protein pVI was used for citrullination experiments (see, e.g., SEQ ID NO:3). Both WT M13 (see, e.g., SEQ ID NO:5) as two recombinant M13 clones (M13 clone 1 and M13 clone 2) (see, e.g., SEQ ID NOS:6 and 7) were used. cDNA inserts of recombinant M13 were cloned in a multiple cloning site downstream from the gene encoding phage coat protein pVI and a GS-linker sequence (see, e.g., SEQ ID NO:4). Minor coat protein pVI contains two arginine amino acids available for conversion to citrulline (indicated in bold). Sequences of the multiple cloning site contribute another two arginine amino acids in the WT M13 clone (four arginines in total). The insert of M13 clone 1 encodes a 28-amino acid peptide that contains three additional arginine amino acids (five arginines in total). The M13 clone 2 polypeptide contains an additional four arginines (six arginines in total). (B) Novagen's T7Select phage vector (see, e.g., SEQ ID NO:8) contains a cloning region at the 3′ end of the gene encoding T7 capsid protein 10B (397 aa). The insert cloned into the T7 vector in T7 S-Tag phage (see, e.g., SEQ ID NO:9) encodes a 15-aa long peptide that contains one arginine amino acid that is displayed 415 times at the capsid of the T7 phage.

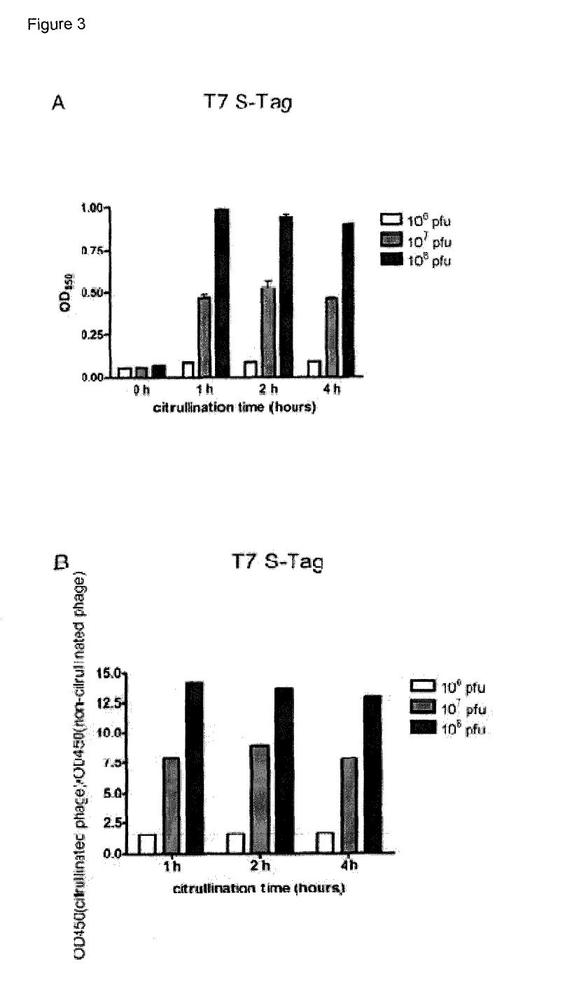

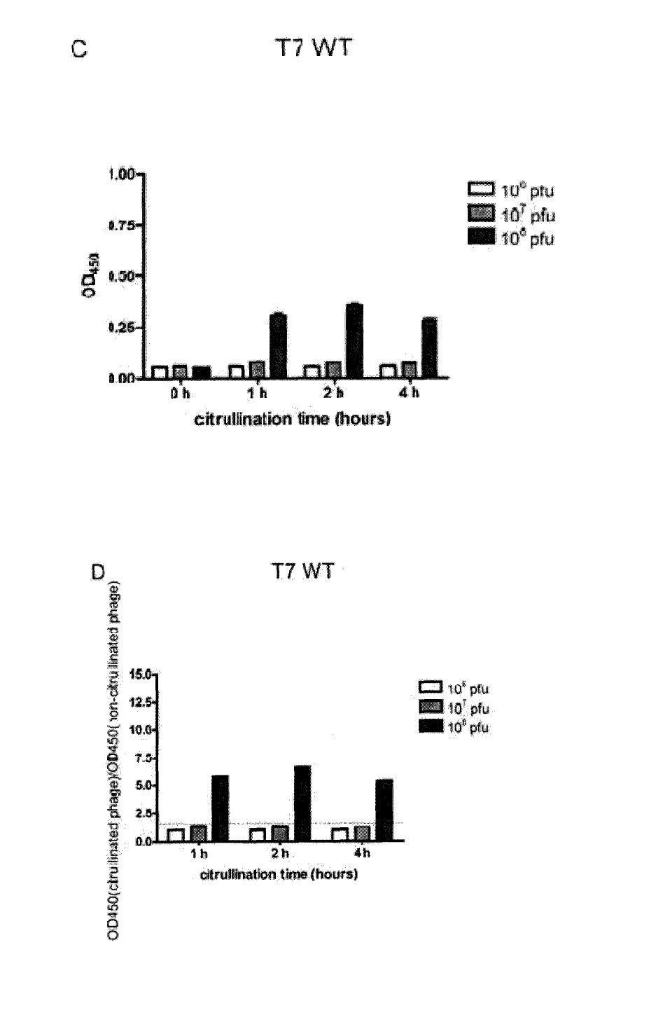

FIG. 3: Citrullination of recombinant and wild-type T7 phage. Recombinant T7 S-Tag and WT T7 phage were citrullinated for different time periods (1, 2 and 4 hours) and the extent of phage citrullination was determined by application of the AMC detection kit. Different amounts of citrullinated and non-citrullinated phage (10⁶, 10⁷ and 10⁸ pfu) were coated per well and the present citrulline amino acids were detected by an anti-citrulline (modified) antibody. The measured OD450 is representative for the extent of citrullination. Citrullination was measured in recombinant (A-B) and WT (C-D) T7 phage. Background reactivity was accounted for by measuring OD450 of non-citrullinated phage (0 hours). In B and D, the ratio of OD450 (citrullinated phage) to OD450 (non-citrullinated phage) is depicted. A ratio of more than 1.5 was considered a positive signal for citrullination. Experiments were performed three times independently.

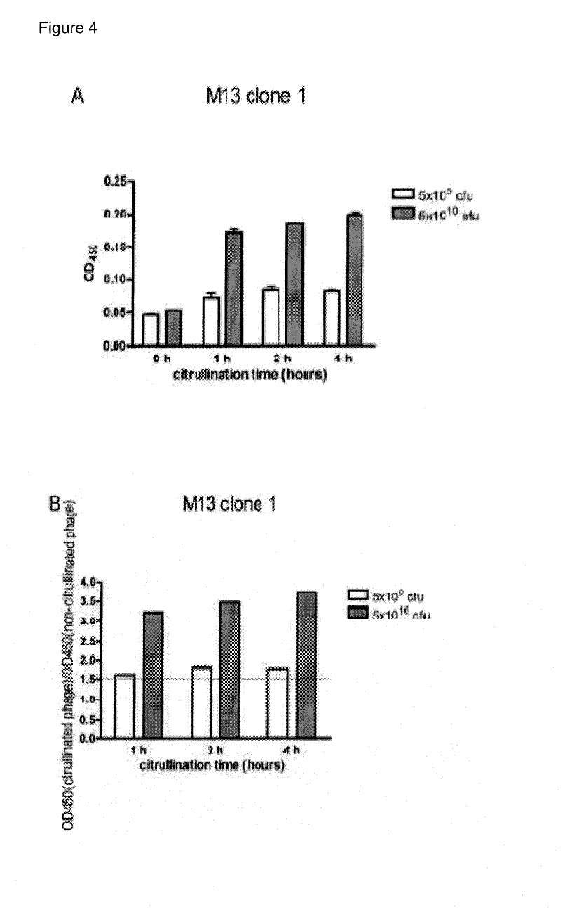

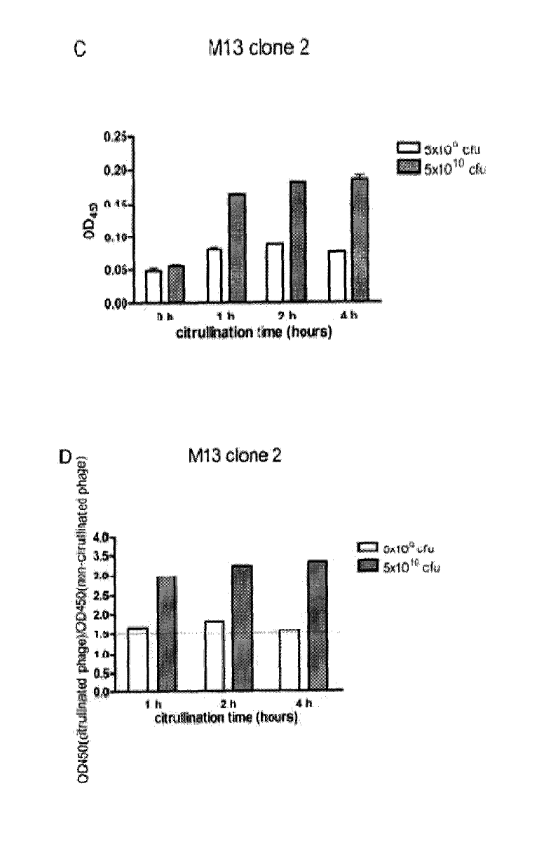

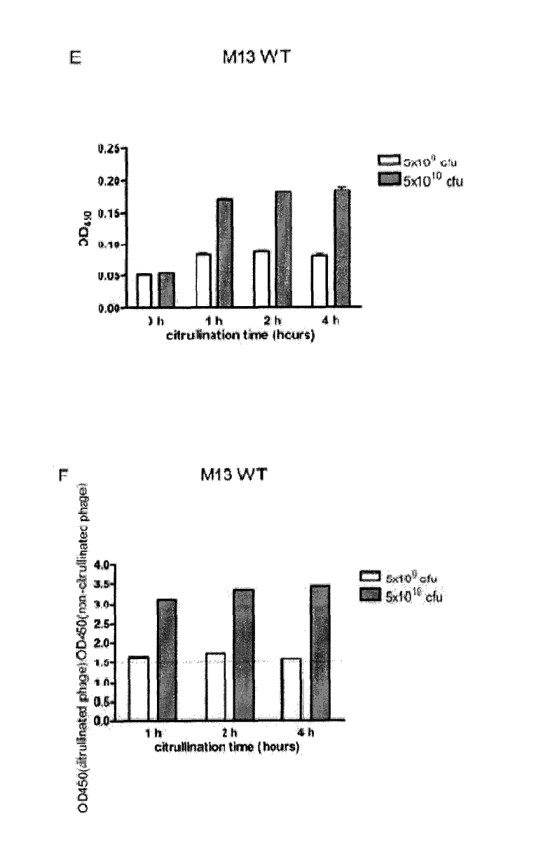

FIG. 4: Citrullination of recombinant and wild-type M13 phage. Recombinant (M13 clone 1 and M13 clone 2) and WT M13 phage were citrullinated for different time periods (1, 2, 4 hours) and the extent of phage citrullination was measured by means of the AMC detection kit. Different amounts of citrullinated and non-citrullinated phage (5×10⁹ and 5×10¹⁰ cfu) were coated per well and the present citrulline amino acids were detected by an anti-citrulline (modified) antibody. The measured OD450 is representative for the extent of citrullination. Citrullination was measured in recombinant (A-D) and WT (E-F) M13 phage. Background reactivity was accounted for by measuring OD450 of non-citrullinated phage (0 hours). In B, D, and F, the ratio of OD450 (citrullinated phage) to OD450 (non-citrullinated phage) is depicted. A ratio of more than 1.5 was considered a positive signal for citrullination. Experiments were independently performed three times.

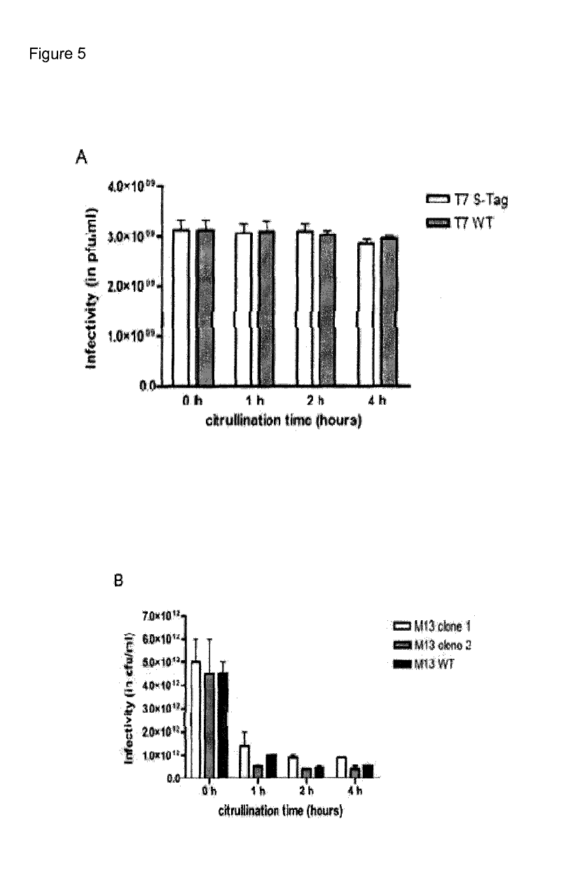

FIG. 5: Effect of citrullination on infectivity of T7 and M13 phage. (A) By performing infection experiments with appropriate E. coli host bacteria, the infection efficiency of citrullinated T7 and M13 phage (1, 2 and 4 hours citrullination) was compared to non-citrullinated phage (0 hours). For T7 phage, citrullination was shown not to have an effect on infection efficiency as the number of infecting phage did not change after citrullination. Obtained titers were within the normal range of T7 phage titers (10⁹-10¹⁰ pfu/ml). (B) For M13 phage, the citrullination of phage particles did result in a decrease of infection titer compared to non-citrullinated phage. The obtained titer for non-citrullinated M13 phage was within the normal range of M13 phage titers. Experiments were independently performed three times.

### DETAILED DESCRIPTION

### Examples

### Materials and Methods to the Examples

### Vectors and Bacterial Strains

M13 and T7 phage display vectors were used for citrullination and infectivity experiments. For M13 filamentous phage experiments, we made use of M13 pVI-display phagemid vectors, which allow covalent attachment of (c)DNA insert products to the C-terminal end of minor phage coat protein pVI allowing display of the peptide/protein products at the phage surface (FIG. 2, Panel A) (Hufton et al., 1999; Jespers et al., 1995). Experiments were performed with the pVI phagemid vector without insert (wild-type (WT) M13 displaying pVI containing four arginine amino acids) as well as with two recombinant phagemid vectors (M13 clone 1 and M13 clone 2). The cDNA insert of M13 clone 1 encoded a 28-amino acid peptide (PGGFRGEFMLGKPDPKPEGKGLGSPYIE (SEQ ID NO:1)), resulting in the display of a recombinant pVI protein containing five arginine amino acids. M13 clone 2 contained a cDNA insert encoding a polypeptide of 121 amino acids (ADDNFSIPEGEEDLAKAIQMAQEQATD TEILERKTVLPSKHAVPEVIEDFLCNFLIKMGMTRTLDCFQSEWYELIQKGVTELRTVGN VPDVYTQIMLLENENKNLKKDLKHYKQAAEYVIF (SEQ ID NO:2)), resulting in the display of a recombinant pVI protein with six arginines (FIG. 2, Panel A). The pVI phagemid display system is characterized by monovalent display of the recombinant pVI (maximally one recombinant protein per phage particle) with a total of five pVI proteins per phage virion (Hufton et al., 1999). E. coli TG1 was used for M13 phage amplification and infection experiments.

For T7 phage display experiments, Novagen's T7Select phage display system was employed. In this system, peptides and proteins are displayed as a fusion to T7 major capsid protein 10B (Novagen, Nottingham, UK). Citrullination experiments were performed with wild-type T7Select415-1b vector without insert and a T7Select415-1b recombinant phage that displays the 15-amino acid S-Tag™ peptide, containing one arginine amino acid, at high-copy number (n=415) at its capsid (FIG. 2, Panel B). E. coli BL21 bacteria were employed for T7 phage amplification and infection experiments.

### M13 and T7 Phage Production

M13 phage particles were produced and purified as described (Somers et al., 2005; Govarts et al., 2007). T7 phage virions were produced according to the manufacturer's recommendations (Novagen).

### In Vitro Citrullination

Phage particles were citrullinated in vitro with rabbit PAD enzyme according to the manufacturer's recommendations (Sigma-Aldrich, Bornem, Belgium) and previous publications (Pratesi et al., 2006; Kinloch et al., 2005). In brief, M13 and T7 phage particles were PEG (polyethylene glycol)-precipitated, after which the phage pellet was resolved in PAD buffer (0.1 M Tris-Cl, pH 7.4, 10 mM CaCl₂, 5 mM DTT) at 2 mg/ml. PAD enzyme was added at 2 U/mg phage (approximately 2U/10¹² cfu M13 phage and 2U/10⁹ pfu T7 phage) followed by incubation at 50° C. for 1, 2 or 4 hours to allow conversion of arginine amino acids into citrulline amino acids. As a negative control, M13 and T7 phage particles were incubated in PAD buffer at 50° C. without addition of PAD enzyme.

Citrullination of phage particles was confirmed by application of the Anti-Citrulline (Modified) Detection Kit (AMC kit, Upstate, Lake Placid, N.Y.) in an ELISA format with coated phage particles. In brief, citrullinated phage particles were PEG-precipitated and the phage pellet was dissolved in PBS (phosphate-buffered saline). Phage particles were coated overnight in PBS at 4° C. in a 96-well plates (Nunc, Roskilde, Denmark). For M13 phage, 5×10⁹ and 5×10¹⁰ phage particles (cfu) were coated per well. As working titers for T7 phage are 100 to 1000 times lower than M13 phage titers, 10⁶, 10⁷ and 10⁸ T7 phage (pfu) were coated per well. After washing with MilliQ, ELISA plates were blocked with TBS (Tris-buffered saline) containing 0.1% ovalbumin followed by incubating the phage-coated plate with 4% paraformaldehyde. Next, the citrulline residues were modified by overnight incubation (at 37° C.) with a strong acid solution containing 2,3 butanedione monoxime and antipyrine (0.25% 2,3-butanedione monoxime, 0.125% antipyrine, 0.25 M acetic acid, 0.0125% FeCl₃, 24.5% H₂SO₄, 17% H₃PO₄), to form ureido group adducts. This modification ensures the detection of citrulline-containing proteins regardless of the neighboring amino acid sequences. After washing with MilliQ and blocking with 3% milk powder in TBS (M-TBS), the wells were incubated with polyclonal rabbit anti-Citrulline (Modified) antibody (1/1000 in M-TBS) for 3 hours at room temperature. Citrulline residues were detected by addition of goat anti-rabbit IgG conjugated to HRP for 1 hour at room temperature (1/5000 in M-TBS), followed by color development with TMB substrate (3, 3′, 5, 5′ tetramethylbenzidine) (Sigma-Aldrich). The reaction was stopped by addition of 2M H2SO₄ and color development was read at 450 nm. Background reactivity was accounted for by measuring OD450 of coated non-citrullinated phage (0 hours). A ratio of OD450 (citrullinated phage) to OD450 (non-citrullinated phage) above 1.5 was considered a positive signal for citrullination.

### Phage Virion Viability and Infectivity Tests

The viability and infectivity of citrullinated phage were determined by counting the number of virions that were able to infect E. coli bacteria after citrullination, resulting in colony or plaque formation (expressed in pfu/ml or cfu/ml). Efficiency of infectivity was compared between non-citrullinated phage (in PAD buffer for 2 hours at 50° C. without PAD enzyme) and phage that were citrullinated for different time periods (1, 2 and 4 hours). Serial dilutions of citrullinated and non-citrullinated M13 phage particles were allowed to infect exponentially growing TG1 bacteria (OD600=0.5) for 30 minutes at 37° C. Bacteria were plated on 2×YT agar plates with selective antibiotic (ampicillin, 100 μg/ml) and resulting colonies were counted for M13 phage titer determination. For determination of the infectivity of citrullinated T7 phage, E. coli BL21 bacteria were mixed with serial dilutions of citrullinated and non-citrullinated T7 phage (in LB medium with supplements 1×M9 salts, 0.4% glucose and 1 mM MgSO₄) followed by plating onto LB agar plates in LB topagar. Resulting plaques were counted for T7 phage titer determination.

### Example 1

### Wild-type and Recombinant T7 and M13 Phage Particles can be Citrullinated In Vitro

Wild-type and recombinant T7 and M13 phage were citrullinated in vitro by incubation with PAD enzyme for different time periods (1, 2 and 4 hours). These citrullinated phage were used in a citrulline-detection ELISA approach with the AMC detection kit to confirm citrullination of the phage particles and peptides displayed by the phage virions (FIGS. 3 and 4). For both T7 (FIG. 3) and M13 phage (FIG. 4), citrullination of phage particles by incubation with PAD enzyme could be confirmed: for at least one of the tested coating concentrations, a ratio of OD450 (citrullinated phage) to OD450 (non-citrullinated phage) of more than 1.5 was detected (FIG. 3, Panels B and D, FIG. 4, Panels B, D and F). For both M13 and T7 phage systems, it was shown that already after 1 hour, the PAD enzyme reached its maximum citrullination level indicated by the absence of an increase in citrullination after an additional incubation period of 1 or 3 hours (FIGS. 3 and 4).

For the recombinant T7 S-Tag phage, citrullination could already be easily detected for 107 coated phage virions (FIG. 3, Panels A and B). For the WT T7 on the other hand, the presence of citrulline amino acids was only measurable when coating 10⁸ phage (FIG. 3, Panel C). The level of citrullination (OD450 ratio around 5.5) (FIG. 3, Panel D) was markedly lower compared to the citrullination level of T7 S-Tag phage (OD450 ratio around 13) (FIG. 3, Panel B), indicating a lower intrinsic citrullination level of WT T7 phage. As the only difference between WT T7 phage and recombinant T7 S-Tag phage is the presence of 415 copies of a peptide containing one arginine at the capsid of the recombinant phage, this large difference in citrullination signal can only be accounted for by the signal generated by the displayed peptide. The difference in citrullination signal between WT and recombinant T7 phage provides definite evidence that peptides displayed at the T7 phage surface can be efficiently citrullinated.

When comparing the citrullination efficiencies of all three M13 phage clones, no OD450 differences could be discerned between WT phage (four arginines), M13 clone 1 (five arginines) and M13 clone 2 (six arginines) (FIG. 4). The equal citrullination levels between recombinant and WT M13 can be explained by the copy-number of phage-displayed peptides: the T7 S-Tag protein displays 415 copies of the S-tag protein on its surface, while the number of M13-phage-displayed peptides is maximally one per phage virion.

### Example 2

### T7 Phage Virions Remain Infective after Citrullination, while M13 Phage Virions Become Less Infective

Whether phage particles retain viability and infectivity after post-translational modification by citrullinating enzymes is the most important prerequisite for the possibility to apply this approach in phage display applications. After confirmation of citrullination, citrullinated and non-citrullinated phage were allowed to infect susceptible bacteria and titers of infecting phage virions were determined based on the number of resulting colonies or plaques (FIG. 5). For T7 phage, citrullination did not have an effect on phage infectivity or viability as the titers of citrullinated and non-citrullinated phage were the same (FIG. 5, Panel A). If citrullinated and non-citrullinated phage can evenly infect efficiently, and thus no growth bias is introduced by in vitro citrullination, this in vitro modification can be applied in T7 phage display biopanning experiments. On the other hand, for both recombinant and WT M13 phage, the infecting phage titer decreased at least five-fold upon citrullination (FIG. 5, Panel B). The decrease in infectivity was comparable for WT M13 and both recombinant M13 clones. This clearly indicates a negative effect of phage coat protein citrullination on M13 phage infectivity. The difference in effect of in vitro citrullination on M13 and T7 phage infection efficiency can be explained by the fact that both phage have completely different bacterial infection mechanisms. T7 phage use their tail fiber proteins to bind and infect bacteria, while M13 phage rely on M13 minor coat protein pIII for efficient infection. If the conversion of present arginine residues into citrulline abrogates the interactions between these phage infection proteins and their binding targets on bacterial cells, infection efficiency is diminished. Indeed, when looking into the amino acid sequence of M13 coat protein pIII (406 amino acids), nine arginine residues are detected. It is thought that the N-terminal region between amino acid 53 and 196 of pIII is essential for successful bacterial infection. As this region contains three arginine amino acids, it is possible that conversion of one or more of these arginines into citrulline has a negative effect on M13 infection efficiency. As the major structural M13 capsid protein pVIII that makes up almost the entire M13 virion capsid except for the ends does not contain an arginine, it is unlikely that citrullination affects phage stability and viability. Mutation experiments in which the essential pIII arginines are replaced by other amino acids to retain infectivity can be performed to allow the application of citrullination in M13 phage display systems. As the decrease of phage infectivity was already maximal after 1 hour of citrullination, this again indicates that 1 hour is sufficient for PAD to citrullinate the present arginines.

### REFERENCES

Alberts B., A. Johnson, J. Lewis, M. Raff, K. Roberts, and P. Walter. Molecular Biology of the Cell. Garland Science, Taylor & Francis Group, 2008.
Anderton S. M. Post-translational modifications of self antigens: implications for autoimmunity. Curr. Opin. Immunol. 2004; 16 (6):753-758.
Armstrong N., N. Adey, S. McConnell, and B. Kay. “Vectors for Phage Display,” in B. Kay, J. Winter, J. McCafferty, editors. Phage Display of Peptides and Proteins. A Laboratory Manual. San Diego: Academic Press, INC, 1996: 35-54.
Asaga H., M. Yamada, and T. Senshu. Selective deimination of vimentin in calcium ionophore-induced apoptosis of mouse peritoneal macrophages. Biochem. Biophys. Res. Commun. 1998; 243 (3):641-646.
Barbas C.F., III. Recent advances in phage display. Curr. Opin. Biotechnol. 1993; 4 (5):526-530.
Cantaert T., L. De Rycke, T. Bongartz, E. L. Matteson, P. P. Tak, A. P. Nicholas, et al. Citrullinated proteins in rheumatoid arthritis: crucial . . . but not sufficient! Arthritis Rheum. 2006; 54 (11):3381-3389.
Chapuy-Regaud S., M. Sebbag, D. Baeten, C. Clavel, C. Foulquier, F. De Keyser, et al. Fibrin deimination in synovial tissue is not specific for rheumatoid arthritis but commonly occurs during synovitides. J. Immunol. 2005; 174 (8):5057-5064.
Crameri R., R. Jaussi, G. Menz, and K. Blaser. Display of expression products of cDNA libraries on phage surfaces. A versatile screening system for selective isolation of genes by specific gene-product/ligand interaction. Eur. J. Biochem. 1994; 226 (1):53-58.
Dente L., C. Vetriani, A. Zucconi, G. Pelicci, L. Lanfrancone, P. G. Pelicci, and G. Cesareni. Modified phage peptide libraries as a tool to study specificity of phosphorylation and recognition of tyrosine-containing peptides. J. Mol. Biol. 1997; 269: 694-703.
Deraos G., K. Chatzantoni, M. T. Matsoukas, T. Tselios, S. Deraos, M. Katsara et al. Citrullination of linear and cyclic altered peptide ligands from myelin basic protein (MBP(87-99)) epitope elicits a Th1 polarized response by T cells isolated from multiple sclerosis patients: implications in triggering disease. J. Med. Chem. 2008; 51 (24):7834-7842.
Govarts C., K. Somers, R. Hupperts, P. Stinissen, and V. Somers. Exploring cDNA phage display for autoantibody profiling in the serum of multiple sclerosis patients: optimization of the selection procedure. Ann. N.Y. Acad. Sci. 2007; 1109:372-384.
György B., E. Toth, E. Tarsca, A. Falus and E. I. Buzas. Citrullination: a post-translational modification in health and disease. Int. J. Biochem. Cell Biol. 2006; 38:1662-1677.
Hill J. A., S. Southwood, A. Sette, A. M. Jevnikar, D. A. Bell, and E. Cairns. Cutting edge: the conversion of arginine to citrulline allows for a high-affinity peptide interaction with the rheumatoid arthritis-associated HLA-DRB1*0401 MHC class II molecule. J. Immunol. 2003; 171 (2):538-541.
Hufton S. E., P. T. Moerkerk, E. V. Meulemans, A. de Bruine, J. W. Arends, and H. R. Hoogenboom. Phage display of cDNA repertoires: the pVI display system and its applications for the selection of immunogenic ligands. J. Immunol. Methods 1999; 231 (1-2):39-51.
Ishida-Yamamoto A., T. Senshu, H. Takahashi, K. Akiyama, K. Nomura, and H. Iizuka. Decreased deiminated keratin K1 in psoriatic hyperproliferative epidermis. J. Invest. Dermatol. 2000; 114 (4):701-705.
Ishigami A., T. Ohsawa, M. Hiratsuka, H. Taguchi, S. Kobayashi, Y. Saito et al. Abnormal accumulation of citrullinated proteins catalyzed by peptidylarginine deiminase in hippocampal extracts from patients with Alzheimer's disease. J. Neurosci. Res. 2005; 80 (1):120-128.
Jacobsson K., A. Rosander, J. Bjerketorp, and L. Frykberg. Shotgun Phage Display—Selection for Bacterial Receptins or other Exported Proteins. Biol. Proced. Online 2003; 5:123-135.
Jespers L. S., J. H. Messens, A. De Keyser, D. Eeckhout, I. Van dB, Y. G. Gansemans, et al. Surface expression and ligand-based selection of cDNAs fused to filamentous phage gene VI. Biotechnology (N.Y.) 1995; 13 (4):378-382.
Kinloch A., V. Tatzer, R. Wait, D. Peston, K. Lundberg, P. Donatien et al. Identification of citrullinated alpha-enolase as a candidate autoantigen in rheumatoid arthritis. Arthritis Res. Ther. 2005; 7 (6):R1421-R1429.
Krumpe L. R., A. J. Atkinson, G. W. Smythers, A. Kandel, K. M. Schumacher, J. B. McMahon, et al. T7 lytic phage-displayed peptide libraries exhibit less sequence bias than M13 filamentous phage-displayed peptide libraries. Proteomics 2006; 6 (15):4210-4222.
Krueger K. E., S. Srivastava. Post-translational protein modifications: current implications for cancer detection, prevention, and therapeutics. Mol. Cell Proteomics 2006; 5 (10):1799-1810.
Li P., H. Yao, Z. Zhang, M. Li, Y. Luo, R. R. Thompson, et al. Regulation of p53 target gene expression by peptidylarginine deiminase 4. Mol. Cell Biol. 2008; 28 (15):4745-4758.
Lundberg K., S. Nijenhuis, E. R. Vossenaar, K. Palmblad, W. J. van Venrooij, L. Klareskog, et al. Citrullinated proteins have increased immunogenicity and arthritogenicity and their presence in arthritic joints correlates with disease severity. Arthritis Res. Ther. 2005; 7 (3):R458-R467.
Marks J. D., H. R. Hoogenboom, T. P. Bonnert, J. McCafferty, A. D. Griffiths, and G. Winter. By-passing immunization. Human antibodies from V-gene libraries displayed on phage. J. Mol. Biol. 1991; 222 (3):581-597.
Mastronardi F. G., D. D. Wood, J. Mei, R. Raijmakers, V. Tseveleki, H. M. Dosch et al. Increased citrullination of histone H3 in multiple sclerosis brain and animal models of demyelination: a role for tumor necrosis factor-induced peptidylarginine deiminase 4 translocation. J. Neurosci. 2006; 26 (44):11387-11396.
McCafferty J., A. D. Griffiths, G. Winter, and D. J. Chiswell. Phage antibodies: filamentous phage displaying antibody variable domains. Nature 1990; 348 (6301):552-554.
Mechin M. C., M. Enji, R. Nachat, S. Chavanas, M. Charveron, A. Ishida-Yamamoto et al. The peptidylarginine deiminases expressed in human epidermis differ in their substrate specificities and subcellular locations. Cell. Mol. Life Sci. 2005; 62 (17):1984-1995.
Musse A. A., J. M. Boggs, and G. Harauz. Deimination of membrane-bound myelin basic protein in multiple sclerosis exposes an immunodominant epitope. Proc. Natl. Acad. Sci. U.S.A. 2006; 103 (12):4422-4427.
Nachat R., M. C. Mechin, H. Takahara, S. Chavanas, M. Charveron, G. Serre et al. Peptidylarginine deiminase isoforms 1-3 are expressed in the epidermis and involved in the deimination of K1 and filaggrin. J. Invest. Dermatol. 2005; 124 (2):384-393.
Nicholas A. P., T. Sambandam, J. D. Echols, and W. W. Tourtellotte. Increased citrullinated glial fibrillary acidic protein in secondary progressive multiple sclerosis. J. Comp. Neurol. 2004; 473 (1):128-136.
Pratesi F., C. Tommasi, C. Anzilotti, D. Chimenti, and P. Migliorini. Deiminated Epstein-Barr virus nuclear antigen 1 is a target of anti-citrullinated protein antibodies in rheumatoid arthritis. Arthritis Rheum. 2006; 54 (3):733-741.
Raijmakers R., J. Vogelzangs, J. L. Croxford, P. Wesseling, W. J. van Venrooij, and G. J. Pruijn. Citrullination of central nervous system proteins during the development of experimental autoimmune encephalomyelitis. J. Comp. Neurol. 2005; 486 (3):243-253.
Rodi D. J., and L. Makowski. Phage-display technology—finding a needle in a vast molecular haystack. Curr. Opin. Biotechnol. 1999; 10 (1):87-93.
Rodi D. J., A. S. Soares, and L. Makowski. Quantitative assessment of peptide sequence diversity in M13 combinatorial peptide phage display libraries. J. Mol. Biol. 2002; 322 (5):1039-1052.
Russel M. Filamentous phage assembly. Mol. Microbiol. 1991; 5 (7):1607-1613.
Sambrook J., E. F. Fritsch, and T. Maniatis. Molecular Cloning: A Laboratory Manual, Cold Spring Harbor, N.Y.: Cold Spring Harbor Laboratory Press, 1989.
Schellekens G. A., B. A. de Jong, F. H. van den Hoogen, L. B. van de Putte, and W. J. van Venrooij. Citrulline is an essential constituent of antigenic determinants recognized by rheumatoid arthritis-specific autoantibodies. J. Clin. Invest. 1998; 101 (1):273-281.
Schmitz R., G. Baumann, and H. Gram. Catalytic specificity of phosphotyrosine kinase Blk, Lyn, c-Src and Syk as assessed by phage display. J. Mol. Biol. 1996; 260: 664-677.
Smith G. P. Filamentous fusion phage: novel expression vectors that display cloned antigens on the virion surface. Science 1985; 228 (4705):1315-1317.
Smith G. P., and V. A. Petrenko. Phage Display. Chem. Rev. 1997; 97 (2):391-410.
Somers V., C. Govarts, N. Hellings, R. Hupperts, and P. Stinissen. Profiling the autoantibody repertoire by serological antigen selection. J. Autoimmun. 2005; 25 (3):223-228.
Soumillion P., L. Jespers, M. Bouchet, J. Marchand-Brynaert, G. Winter, and J. Fastrez. Selection of beta-lactamase on filamentous bacteriophage by catalytic activity. J. Mol. Biol. 1994; 237 (4):415-422.
Stolz J., A. Ludwig, and N. Sauer. Bacteriophage lambda surface display of a bacterial biotin acceptor domain reveals the minimal peptide size required for biotinylation. FEBS Lett. 1998; 440: 213-217.
Tarsca E., L. N. Marekov, G. Mei, G. Melino, S. C. Mee, and P. M. Steiner. Protein unfolding by peptidylarginine deiminase. J Biol. Chem. 1996; 48: 30709-30716.
van Boekel M. A., and W. J. van Venrooij. Modifications of arginines and their role in autoimmunity. Autoimmun. Rev. 2003; 2 (2):57-62.
van Venrooij W. J., J. J. van Beers, and G. J. Pruijn. Anti-CCP Antibody, a Marker for the Early Detection of Rheumatoid Arthritis. Ann. N.Y. Acad. Sci. 2008; 1143:268-285.
Vossenaar E. R., T. J. Smeets, M. C. Kraan, J. M. Raats, W. J. van Venrooij, P. P. Tak. The presence of citrullinated proteins is not specific for rheumatoid synovial tissue. Arthritis Rheum. 2004; 50 (11):3485-3494.
Webster R. Biology of the Filamentous Bacteriophage. In: B. Kay, J. Winter, and J. McCafferty, editors. Phage Display of Peptides and Proteins: A Laboratory Manual. San Diego, Calif.: Academic Press, INC, 1996: 1-20.
Yao H., P. Li, B.J. Venters, S. Zheng, P.R. Thompson, B. F. Pugh et al. Histone Arg modifications and p53 regulate the expression of OKL38, a mediator of apoptosis. J. Biol. Chem. 2008; 283 (29):20060-20068.

1. An infective phage, displaying a peptide comprising arginine, wherein at least one arginine of the displayed peptide is citrullinated.

2. The infective phage according to claim 1, wherein said infective phage is a T7 phage.

3. A method of isolating a polypeptide binding citrullinated protein, the method comprising:
utilizing the infective phage according to claim 1 to isolate polypeptide binding citrullinated protein.

4. The method according to claim 3, wherein said polypeptide is an antibody against citrullinated proteins.

5. The method according to claim 4, wherein said antibody against citrullinated proteins is a rheumatoid arthritis autoantibody.

6. A method of citrullinating a peptide displaying phage, without affecting the infective capacity of the phage.

7. The method according to claim 6, wherein said peptide displaying phage is a T7 phage.

8. The method according to claim 6, wherein said method is carried out in vitro.

9. The method according to claim 6, wherein said citrullination is carried out by treatment with a Ca²⁺-dependent peptidyl arginine deaminase.

10. A method of isolating polypeptides binding citrullinated proteins, the method comprising:
utilizing the phage of claim 2 to isolate a polypeptide binding citrullinated protein.

11. The method according to claim 3, wherein the polypeptide is an antibody against citrullinated protein.

12. The method according to claim 4, wherein the antibody against citrullinated protein is a rheumatoid arthritis autoantibody.

13. A method of citrullinating a peptide displaying phage without affecting the peptide displaying phage's infective capacity, the method comprising:
utilizing in the method a Ca2+-dependent peptidyl arginine deaminase.

14. The method according to claim 13, wherein the peptide displaying phage is a T7 phage.

15. The method according to claim 13, wherein the method is carried out in vitro.

16. The method according to claim 14, wherein the method is carried out in vitro.

17. A method of isolating a rheumatoid arthritis autoantibody, the method comprising:
utilizing the infective phage of claim 2 to isolate a rheumatoid arthritis autoantibody against citrullinated protein.
